(19) United States
(12) Patent Application Publication
(10) Pub. No.: US 2025/0283161 A1
(43) Pub. Date: Sep. 11, 2025

(54) PREPARATION OF NUCLEIC ACID SAMPLES FOR SEQUENCING

(71) Applicant: GRAIL, LLC, Menlo Park, CA (US)

(72) Inventors: Matthew LARSON, San Francisco, CA (US); Curtis TOM, San Francisco, CA (US); Sarah STUART, Pacifica, CA (US); Yiqi ZHOU, San Mateo, CA (US)

(21) Appl. No.: 18/267,769

(22) PCT Filed: Dec. 18, 2021
(86) PCT No.: PCT/US2021/064248

§ 371 Date: Jun. 15, 2023

Related U.S. Application Data
(60) Provisional application No. 63/127,566, filed on Dec. 18, 2020.

(51) Int. Cl. C12Q 1/6855

(52) U.S. Cl. CPC C12Q 1/6855

(57) ABSTRACT

Compositions and methods are provided for amplifying nucleic acids, including cell free nucleic acid fragments, in preparation for sequencing. Methods are provided for making circularized nucleic acid templates having the structure [T]-[PS1]-[L]-[PS2] or [PS1]-[L]-[PS2]-[T′], where (a) T is a target nucleic acid and T′ is a complement to a target nucleic acid; (b) each of PS1 and PS2 is a nucleic acid primer site; (c) L is a linker having a primer extension reaction terminating organic molecule; and the structure is circularized by binding a 5′ end thereof to a 3′ end thereof. Target sequences in the circularized templates are amplified by binding to PS1 a primer complimentary to PS1 and binding to PS2 a primer complimentary to PS2 and copying the target sequences by a primer extension reaction. Advantages include a reduction in ligation steps, which can result in fewer clean up steps and improved library conversion efficiency.

### CROSS-REFERENCE TO RELATED APPLICATIONS

This application claims the benefit of priority of U.S. Provisional patent application No. 63/127,566, filed on Dec. 18, 2020, the disclosure of which is incorporated herein by this reference in its entirety.

### TECHNICAL FIELD

The presently disclosed subject matter is directed to compositions and methods for preparation of nucleic acid samples for sequencing.

### BACKGROUND

Methylation of cytosines in DNA is an increasingly important diagnostic marker for a variety of diseases and conditions. For example, DNA methylation profiling has been used as a diagnostic tool for detection, diagnosis, and/or characterization of cancer. These analyses often use fragmented DNA from bodily fluids (cfDNA). Sequencing to identify methylated bases typically involves a conversion step in which unmethylated cytosines are converted to uracils, followed by addition of sequencing adapters. Addition of sequencing adapters typically involves two ligation steps, which can reduce conversion efficiency, and which adds additional cleanup steps. Such an approach reduces overall library conversion efficiency.

Accordingly, there is a need in the art for processes which simplify preparation of DNA libraries and improve efficiency.

### SUMMARY

The presently disclosed subject matter is directed to a method of making a circularized template, the method in various embodiments including: (a) providing a sample of single stranded nucleic acid fragments; (b) ligating to or copying into each of the nucleic acid fragments a fragment having the structure [PS]-[L]-[PS], where PS is a primer site and L is a linker; (c) circularizing, and/or any one or combination of such aspects or other aspects disclosed herein.

In some aspects, the disclosed subject matter includes a circularized nucleic acid template having the structure [T]-[PS1]-[L]-[PS2], wherein: (a) T is a target nucleic acid sequence; (b) each of PS1 and PS2 is a nucleic acid primer site; (c) L is a linker including a primer extension reaction terminating organic molecule, e.g., the linker L joins PS1 and PS2 and includes an organic molecule that terminates polymerization in a primer extension reaction; and (d) the structure is circularized by binding a 5′ end thereof to a 3′ end thereof, and/or any one or combination of such aspects or other aspects disclosed herein.

In various embodiments, the disclosed subject matter includes a circularized nucleic acid template having the structure [PS1][L]4-[PS2]-[T′], wherein: (a) T′ is a complement of a target nucleic acid sequence; (b) each of PS1 and PS2 is a nucleic acid primer site; (c) L is a linker that includes a primer extension reaction terminating organic molecule; and (d) the structure is circularized by binding a 5′ end thereof to a 3′ end thereof and/or any one or combination of such aspects or other aspects disclosed herein.

In some aspects, the present disclosure is directed to a method of amplifying a target sequence, the method including: (a) providing circularized template; (b) binding to PS1 a primer complimentary to PS1 and binding to PS2 a primer complimentary to PS2; (c) copying the target sequence by a primer extension reaction using a polymerase, and/or any one or combination of such steps or other aspects disclosed herein.

In various instances, the present disclosure is directed to a method of making a circularized template having the structure [T]-[PS1]-[L]-[PS2], the method including: (a) providing a [PS1]-[L]-[PS2] strand; (b) providing a [T] strand; (c) ligating the [PS1]-[L]-[PS2] strand to the [T] strand to produce a [T]-[PS1]-[L]-[PS2] strand; (d) circularizing [T]-[PS1]-[L]-[PS2] strand to produce a circularized [T]-[PS1]-[L]-[PS2], and/or any one or combination of such steps or other aspects disclosed herein, (method circularized template approach #1-new) In some instances the present disclosure is directed to a method of making a circularized template having a structure [PS1]-[L]-[PS2]-[!″ ], the method including: (a) providing a [T] strand; (b) providing a [PS1]-[L]-[PS2] strand (c) ligating to a 3′ end of the [T] strand a [PS2′] strand complementary to the [PS2] of the [PS1]-[L]-[PS2] strand; (d) annealing the [PS1]-[L]-[PS2] strand to the ligated [T]-[PS2′]; (d) extending the [PS1]-[L]-[PS2] strand by a primer extension reaction using a polymerase to produce a [PS1]-[L]-[PS2]-[T] strand, wherein [T′] is a complement of the [T] strand; and (e) circularizing the [PS1]-[L]-[PS2]-[T′] strand to produce a circularized [PS1]-[L]-[PS2]-[T′].

In some embodiments, the present disclosure is directed to a single stranded nucleic acid having the structure [PS1]-[L]-[PS2], wherein each of PS1 and PS2 is a nucleic acid primer site and L is a linker including a primer extension reaction terminating organic molecule.

In some aspects, the present disclosure is directed to a kit including: (a) 5′ phosphorylated single stranded primer site oligonucleotide having a blocked 3′ end; and (b) a single stranded nucleic acid having the structure [PS1]-[L]-[PS2], wherein each of PS1 and PS2 is a nucleic acid primer site, L is a linker that joins [PS1] and [PS2] and includes a primer extension reaction terminating organic molecule, and [PS2] is complementary to the primer site oligonucleotide having a blocked 3′ end.

In some embodiments, the linker comprises a poly alkylene glycol linker, a polyethylene glycol linker, a polyalkylene glycol linker terminated with a phosphate group, or a polyethylene glycol linker terminated with a phosphate group.

In various embodiments, the target nucleic acid sequence [T] comprises a single-stranded DNA molecule in which cytosines have been modified or converted. In other embodiments, the target nucleic acids sequence [T] comprises a single-stranded DNA molecule in which cytosines have been converted to uracils.

### BRIEF DESCRIPTION OF THE DRAWINGS

FIG. 1A shows circularized ssDNA with the structure: [T]-[PS]-[L]-[PS] or [PS]-[L]-[PS]-[T′], where a linker [L] joins two primer site [PS] components and includes a primer extension reaction terminating organic molecule, and where [PS] components are joined to a target sequence [T] or to a complement of a target sequence [T′].

FIG. 1B is a diagram illustrating a [PS1]-[L]-[PS2] strand of the disclosed subject matter which includes a first adapter [PS1], a second adapter [PS2], and a linker [L] that includes a primer extension reaction terminating organic molecule and links a 3′ end of the first adapter to a 5′end of the second adapter.

FIG. 1C is a diagram illustrating a linear [T]-[PS1]-[L]-[PS2] strand of the disclosed subject matter, which includes the [PS1]-[L]-[PS2] strand of FIG. 1B joined at 5′ end to a 3′ end of a target strand.

FID. 1D is a diagram illustrating a linear [PS1]-[L]-[PS2]-[T] strand of the disclosed subject matter, which includes the [PS1]-[L]-[PS2] strand of FIG. 1B joined at 3′ end to a 5′ end of a complement of a target strand [T′].

FIGS. 2A and 2B illustrate a method of the disclosed subject matter that includes primer site ligation, primer extension of [PS1]-[L]-[PS2] 102 of FIG. 1B, and amplification.

FIG. 3 illustrates an alternative embodiment in which the primer site ligation and primer extension reactions in Steps C, D and E of FIG. 2A are replaced with direct ligation of [PS1]-[L]-[PS2] 102 of FIG. 1B to the target strand.

FIG. 4 is a graph of a High Sensitivity NGS Fragment Analyzer run illustrating a comparison of cfDNA prepared in an amplification method using either the [PS1]-[L]-[PS2] strand 102 extension primer (designated “Circ extend primer”) or a traditional extension primer where the cfDNA is not circularized (designated “GrailMethylSeq”), and where the cfDNA has received only one of two clean up steps.

FIG. 5 is a graph of a High Sensitivity NGS Fragment Analyzer run of a methylation library that was prepared by circularization approach using Circ extend primer.

### DETAILED DESCRIPTION

As used herein the following terms have the meanings given:

“Amplification” means copying a strand of DNA to produce a complementary strand. Amplification may be thermally mediated or may be isothermal. Amplification may, for example, be accomplished by using a polymerase in a primer extension reaction to copy a target strand.

“Bodily fluid” means any bodily fluid containing DNA, including without limitation, whole blood, circulating blood, a blood fraction, serum, or plasma, aqueous humor, ascites, bile, cerebral spinal fluid, chyle, gastric juices, intestinal juices, lymphatic fluid, pancreatic juices, pericardial fluid, peritoneal fluid, pleural fluid, saliva, spinal fluid, sputum, stool or other intestinal waste fluids, sweat, tears, and/or urine.

“cfNA” means extracellular nucleic acids, and “cfDNA” means extracellular DNA, found in a bodily fluid.

“Input sample” refers to a processed sample of fragmented DNA. The term “input sample” is used to distinguish from a “sample” which refers to a biological sample obtained from a subject. A sample from a biological subject is processed to prepare an input sample, e.g., by purifying cfDNA from the sample and/or fragmenting the sample if needed. Fragmented DNA and cfDNA is referred to herein as “target nucleic acid” “target DNA,” “target strand” or “target.” Nevertheless, it should be noted that in one aspect of the disclosed subject matter, the input sample and the sample may be the same, i.e., the method may be used with a “dirty sample” or an “unpurified sample.”

“Target disease” means a disease, condition, or target for which an assay or test is being performed, e.g., a target disease may be cancer generally, a specific class of cancers, a specific cancer type, a specific cancer stage, combinations of the foregoing, or any other disease or condition or combination of diseases of conditions for which a methylation analysis may produce informative information.

Throughout the specification, the applicants make use of the formula [x]-[y]-[z]-[ . . . ]. to describe various structures, e.g., [T]-[PS]-[L]-[PS] and [PS]-[L]-[PS]-[T′], including primer site [PS], target nucleic acid [T], complement to target nucleic acid [T′], linker [L], and adapter [A], It will be appreciated that these formulae are not intended to limit the components of the structures described—e.g., one or more additional components may be included. For example, in [T]-[PS]-[L]-[PS] or [PS]-[L]-[PS]-[T′], one or more additional components may be included at either end of the structure or between a [PS] and [T/T′], between a [PS] and [L], or any combination of the foregoing.

### Description

In various aspects, the disclosed subject matter provides methods, compositions, reagents and kits for preparing a fragmented DNA library for sequencing. The method makes use of a circularized ssDNA template that can be used in an amplification reaction to produce linear copies. The linear copies may proceed to other steps in a library preparation workflow and/or into analytical steps, e.g., processes to determine the sequence of the target. Advantages of the disclosed subject matter include a reduction in ligation steps, which can result in fewer clean up steps and improved overall library conversion efficiency.

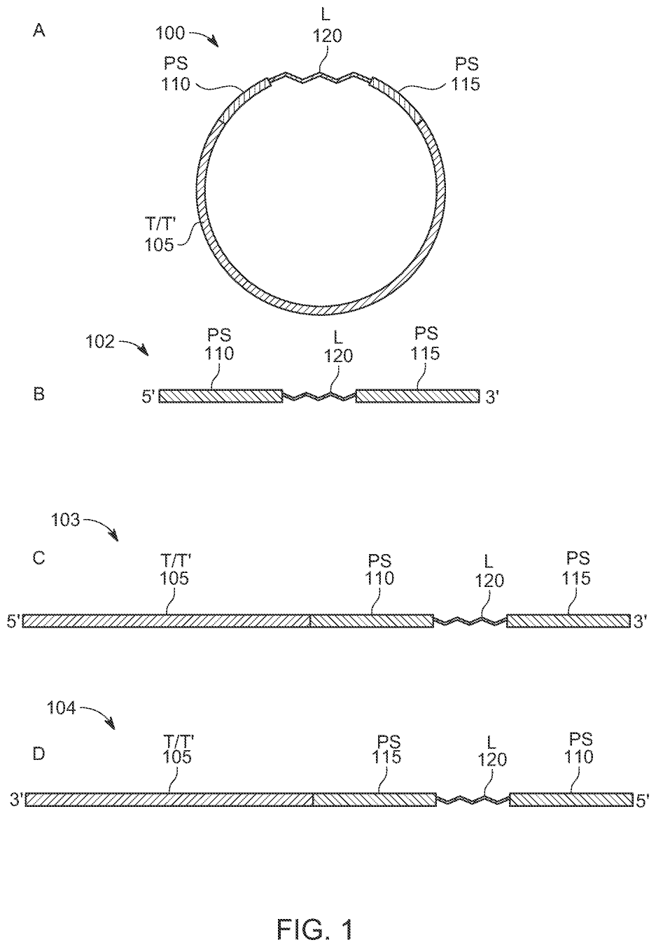

FIG. 1A shows circularized ssDNA 100 with the structure: [T]-[PS]-[L]-[PS] or [PS]-[L]-[PS]-[T′], where linker 120 that includes a primer extension reaction terminating organic molecule joins the two [PS] components which are joined to target sequence [T] or complement to target sequence [T′]. Circularized ssDNA 100 includes a target/complement to target 105, a first primer site 110 at a 3′ or 5′ end of the target, a second primer site 115 at the other end of target 105, and a linker 120.

Linker 120 may comprise any of a variety of primer extension reaction terminating organic molecules that join a 3′ end of a nucleic acid chain to a 5′ end of another nucleic acid chain. The linker 120 terminates polymerization in a primer extension reaction. For example, in one aspect, the linker comprises a polyalkylene glycol molecule terminated with a phosphate group. In one embodiment, the linker comprises a polyethylene glycol molecule terminated with a phosphate group. The polyethylene glycol moiety may, for example, have from 2 to 20 polyethylene glycol units. In one instance, the linker comprises Int Spacer 18 (ISpI8), a hexa-ethyleneglycol linker available from Integrated DNA Technologies, Inc. (Coraville, IA):

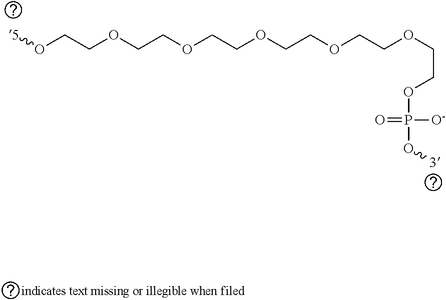

In one embodiment, the linker comprises a phosphonamidite molecule. The phosphonamidite molecule may, for example, have from two to twenty carbon atoms. In one aspect, the linker comprises a three carbon phosphonamnidite molecule available from Integrated DNA Technologies, Inc. (Coraville, IA):

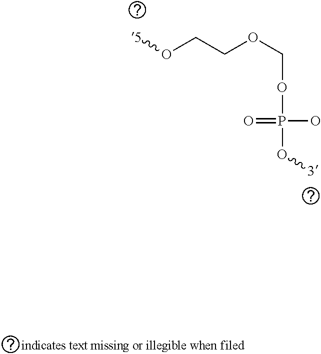

In one version of the embodiments, the linker comprises a glycol molecule. The glycol molecule may, for example, have from two to twenty carbon atoms. In one instance, the linker comprises a six-carbon glycol molecule available from Integrated DNA Technologies, Inc. (Coraville, IA):

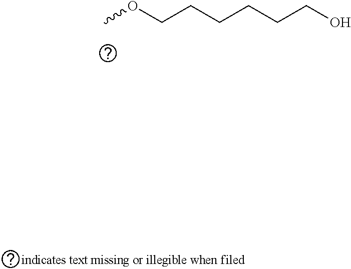

In some versions, the linker comprises one or more 1′,2′-Dideoxyribose molecules or similar abasic molecules. The 1′,2′-Dideoxyribose molecule may, for example, have from two to twenty 1′,2′-Dideoxyribose units. In one aspect, the linker comprises a 1′,2′-Dideoxyribose molecule available from Integrated DNA Technologies, Inc. (Coraville, IA):

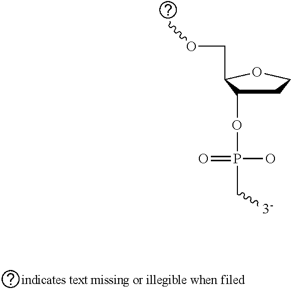

In various embodiments, the linker comprises a triethylene glycol molecule terminated with a phosphate group. In one instance, the linker comprises a triethylene glycol molecule available from Integrated DNA Technologies, Inc. (Coraville, IA)

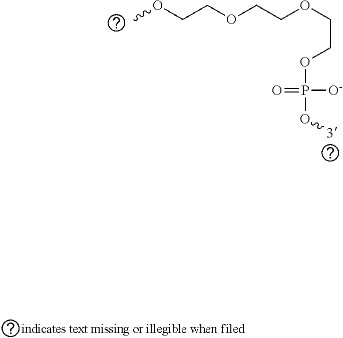

In one aspect, the linker 120 comprises multiple insertions of a primer extension reaction terminating organic molecule. In one instance, the linker 120 comprises multiple insertions of a combination of different organic molecules that join a 3′ end of a nucleic acid chain to a 5′ end of another nucleic acid chain and that terminate polymerization in a primer extension reaction. In a nonlimiting example, the linker 120 may comprise multiple insertions of one or a combination of a polyalkylene glycol molecule, a polyalkylene glycol molecule terminated with a phosphate group, a polyethylene glycol molecule, a polyethylene glycol molecule terminated with a phosphate group, a phosphonamidite molecule, a glycol molecule, a 1′,2′-Dideoxyribose molecule, or a triethylene glycol molecule terminated with a phosphate group.

In some versions, the linker 120 further comprises one or more intervening oligonucleotides connecting multiple insertions of the organic molecules that join a 3′ end ofa nucleic acid chain to a 5′ end of another nucleic acid chain and terminate polymerization in a primer extension reaction. By way of non-limiting example, in one aspect, the linker 120 is an Int Spacer 18 molecule bonded at its 3′ end to the 5′ end of an intervening oligonucleotide, the intervening oligonucleotide bonded at its 3′ end to the 5′ end of a second Int Spacer 18 molecule. The length of the one or more intervening oligonucleotides can range from 2 nucleotides in length to 100 nucleotides in length, from 3 nucleotides in length to 20 nucleotides in length, or from four nucleotides in length to 10 nucleotides in length. In one example, the length of the intervening oligonucleotide is six nucleotides or more.

FIG. 18 is a diagram illustrating a [PS]-[L]-[PS] strand 102 of the disclosed subject matter, which includes a first adapter 110, a second adapter 115 and a linker 120 linking a 3′ end of adapter 110 to a 5′end of adapter 115. Strand 102 may also be referred to herein as Circ extend primer or extension primer. The term adapter as it is used herein refers to a single-stranded or double-stranded oligonucleotide that can be ligated to the ends of other DNA, cfDNA, or RNA molecules. Adaptors can provide primer sites in sequencing.

The [PS]-[L]-[PS] strand 102 of the disclosed subject matter can be generated using any method known to those of ordinary skill in the art. In one non-limiting example, a 5′ DMT (dimethoxytrityl) protected oligonucleotide (N 1) of interest may be attached at the 3′ position to a 5 micron controlled pore glass bead (CPG) or Polystyrene (PS) bead. A non-limiting example of such a N1 oligonucleotide includes DMT-dT-CPG (Sigma-Aldrich, Inc., St. Louis, MO). The dimethoxytrityl group may then be deprotected using trichloroacetic acid (TCA) leaving a 5′ reactive hydroxyl group. Next, the CPG bound oligonucleotide may be washed with a polar aprotic solvent, for example, acetonitrile for 30 seconds.

Next, a 5′-DMT protected Internal Spacer 18 molecule may be reacted with the 5′ exposed hydroxyl group of N1. In this reaction, condensation may occur between the exposed 5′-hydroxyl of N1 and the 3′-phosphate group of Internal Spacer 18, which may form a covalent bond. Subsequently, the products may be washed using a polar aprotic solvent, for example, acetonitrile for 30 seconds. Next, the 5′-DMT group may be removed from the 5′-end of the Internal Spacer 18 using the standard TCA method, which may leave a reactive 5′-hydroxyl group.

A 5′-DMT protected nucleotide (N2) may then be reacted with the CPG—³ dT⁵—3 IntSpacer18-OH⁵ chain, where the “OH” may be an exposed reactive 5′-hydroxyl group. The 5′-hydroxyl group of the Internal Spacer 18 may react with the 3′-hydroxyl group of the N2 5′-DMT-protected nucleotide in a condensation reaction, which may form a covalent bond with the linker at the 3′ carbon of the N2 nucleotide. This condensation reaction may produce a CPG—³N1⁵—³ Linker⁵—N2 chain. Subsequently the above steps may be repeated starting with 5′-DMT deprotection of the N2 nucleotide until the desired [PS]-L-[PS] strand 102, is achieved.

The method of the disclosed subject matter makes use of the [PS]-[L]-[PS] strand 102 for producing the circularized [T]-[PS]-[L]-[PS]. The [PS]-[L]-[PS] strand 102 introduces both 5′ and 3′ primer sites 110 and 115 to the target nucleic acid 105. The [PS]-[L]-[PS] strand 102 includes a linker molecule that joins the [PS] components. The [PS]-[L]-[PS] strand 102 may be joined to a 3′ or 5′ end of a target nucleic acid 105. For example, the [PS]-[L]-[PS] strand 102 may be joined to target 105 by ligation or by primer chain extension using a polymerase.

FIG. 1C is a diagram illustrating linear [T]-[PS]-[L]-[PS] strand 103 of the disclosed subject matter, which includes [PS]-[L]-[PS] strand 102 of the disclosed subject matter joined at 5′ end to a 3′ end of target 105. The [T]-[PS]-[L]-[PS] strand 103 can be circularized to form the circularized ssDNA 100 illustrated in FIG. 1A.

FIG. 1D is a diagram illustrating linear [PS]-[L]-[PS]-[T′] strand 104 of the disclosed subject matter, which includes [PS]-[L]-[PS] strand 102 of the disclosed subject matter joined at 3′ end to a 5′ end of the complement to target 105 [T′]. The [PS]-[L]-[PS]-[T′] strand 104 can be circularized to form the circularized ssDNA 100 illustrated in FIG. 1A.

### Method

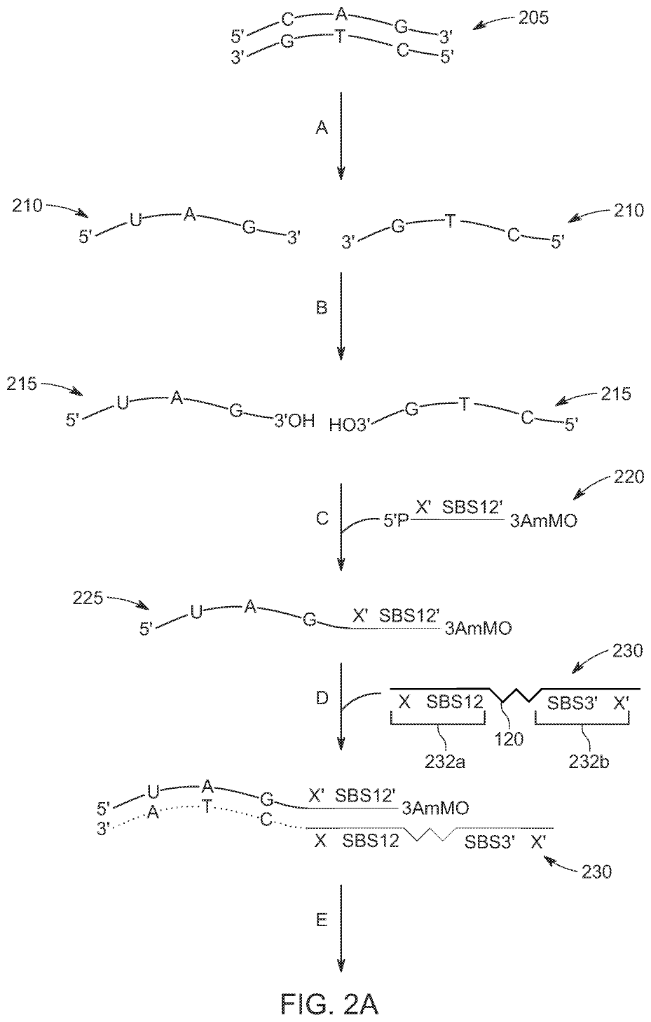

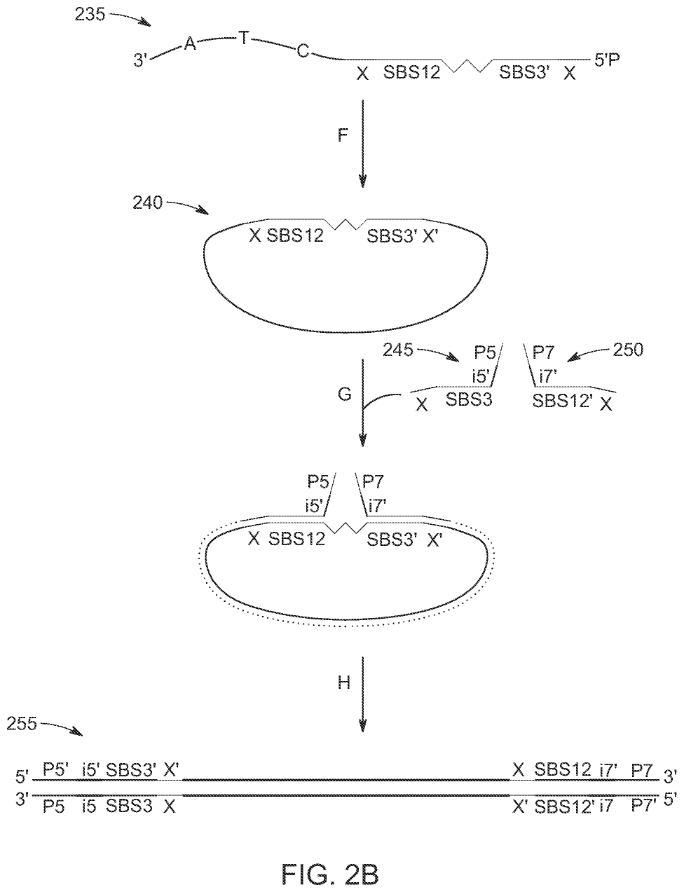

FIGS. 2A and 2B illustrate a method of the disclosed subject matter.

In Step A, double-stranded input nucleic acid fragments 205, undergo conversion, in which the strands are denatured and unmodified cytosines are converted to uracils. Step A yields denatured, converted single-stranded DNA fragments 210.

In Step B, the ends of fragments 210 are prepared for ligation, e.g., using T4 polynucleotide kinase (T4 PNK), which catalyzes the transfer of the y-phosphate of ATP to the 5′ terminus and removes the 3′ phosphate from the single stranded fragments 210 to yield fragments 215.

In Step C, primer site strands 220 are ligated to the 3′ end of fragments 215, yielding [T]-[PS] strands 225. The primer site strands 220 may be introduced as 5′ phosphorylated single stranded strands with blocked 3′ ends. A variety of modifiers are available to block the 3′ ends of the primer site strands 220. Several such modifiers are available from Integrated DNA Technologies, Inc., Coraville, IA. In one aspect, the blocker is 3AmM0, a 3′ amino modifier, available from Integrated DNA Technologies, Inc. (Coraville, IA) having the following structure:

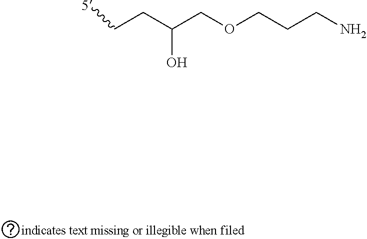

Ligation may in certain embodiments be enzymatically mediated, e.g., using CIRCLIGASE (discussed in more detail below). A clean-up step may be included to prepare the [T]-[PS] strands 225 for Step D.

In StepD, [PS]-[L]-[PS] strands 102 (shown as 230 in FIG. 2A) are introduced. The [PS]-[L]-[PS] strands 230 include primer sites 232a and 232b separated by linker 120. Primer site 232a operates as a primer in this step, annealing to the complementary primer site on [T]-[PS] strand 225. Once annealed, [PS]-[L]-[PS] strands 230 may be extended via primer extension reaction using a polymerase to yield [PS]-[L]-[PS]-[T′] strands 235 (see FIG. 2B).

In Step E, clean-up steps may be performed, optionally including degradation of bisulfite treated template where bisulfite conversion method has been utilized to convert unmethylated cytosines to uracils, e.g., with USER (uracil-specific excision reagent) enzyme (available from New England Biolabs, Inc., Ipswich, MA). In one aspect of the disclosed subject matter, primer extension in Step E is conducted using linear amplification, which can be expected to dilute out the original template, rendering degradation of the original template unnecessary.

In Step F, [PS]-[L]-[PS]-[T′] strands 235 are circularized, yielding circularized [PS]-[L]-[PS]-[T′] templates 240. Circularization may be accomplished using a variety of techniques. In one aspect, circularization is accomplished using a ssDNA ligase, such as CIRCLIGASE ssDNA Ligase or CIRCLIGASE II ssDNA Ligase (discussed in more detail below). Circularization reactions using CIRCLIGASE ssDNA Ligase or CIRCLIGASE II ssDNA Ligase make use of an ssDNA 5′-phosphate and 3′-hydroxyl groups to circularize DNA.

In Step G, forward primers 245 and reverse primers 250 are annealed to circularized [PS]-[L]-[PS]-[T′] templates 240, and a primer extension reaction is used to generate linearized [A]-[T]-[A] amplicons 255. Forward primers 245 and reverse primers 250 may include various adapter elements useful for downstream workflow steps, such as sequencing steps.

The primers are preferably sequencing primers, such as primers for use with Illumina sequencers.

In one example, the forward primers 245 have the following structure, 5′ to 3′:
[ADAPT]-[IND]-[SEQ-PRIMER]-[TARG-PRIMER]
[ADAPT] is an adapter, such as the Illumina P5 adapter, 5′ AAT GAT ACG GCG ACC ACC GA 3′ (SEQ ID NO: 1);
[IND] is a unique sample identifier sequence, such as an Illumina i7 index;
[SEQ-PRIMER] is a sequencing primer, such as an Illumina SBS12 primer ACA CTC TTT CCC TAC ACG ACG CTC TTC CGA TCT (SEQ ID NO: 2);
[TARG-PRIMER] is a target primer complementary to a target nucleic acid sequence in the circularized [PS]-[L]-[PS]-[T′] templates 240.

In one example, the reverse primers 250 have the following structure, 5′ to 3′:
[ADAPT]-[IEND]-[SEQ-PRIRMER]-[TARG-PRIMER]
[ADAPT] is an adapter, such as the Illumina P7 adapter, 5′ CAA GCA GAA GAC GGC ATA CGA 3 (SEQ ID NO: 3);
[lND] is a unique sample identifier sequence, such as an Illumina i5 index;
[SEQ-PRIMER] is a sequencing primer, such as an Illumina SBS3 primer, GTG ACT GGA GTT CAG ACG TGT GCT CTT CCG ATC T (SEQ ID NO: 4);
[TARG-PRIMER] is a target primer complementary to a target sequence in the circularized [PS]-[L]-[PS]-[T′] templates 240.

In Step H, linearized [A]-[T]-[A] amplicons 255 are cleaned up and prepared for analysis, e.g., for sequencing.

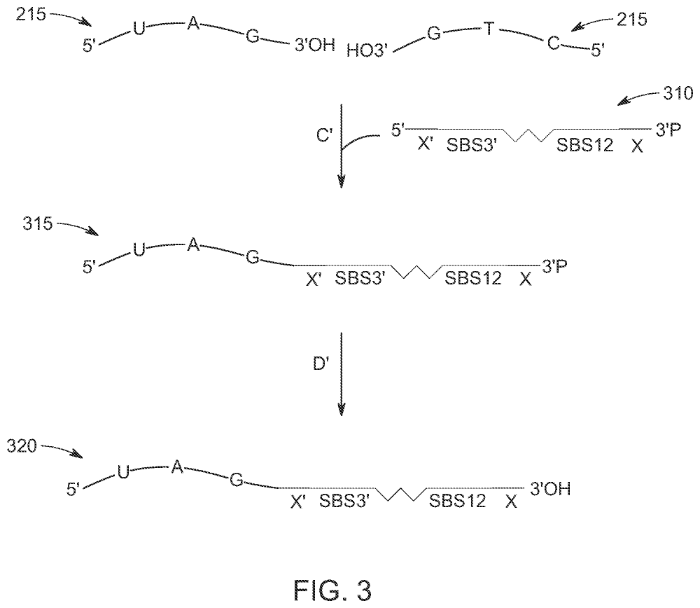

FIG. 3 illustrates an alternative embodiment in which the primer site ligation and primer extension reactions in Steps C, D and E are replaced with a ligation process in which [PS]-L-[PS2] strands 102 are ligated directly to the target strand, illustrated in FIG. 3 as Steps C′ and D′. Thus, the method described in FIG. 3 may, in one aspect, proceed in the following order: Steps A, B, C′, D′, F, G, H, where Steps A, B, F, G and EH are as described above, and Steps C′ and D′ are as follows.

In Step C′, [PS]-[L]-[PS]102 (shown as 310 in FIG. 3) is introduced and ligated to a 3′ end of target fragments 215 to yield [T]-[PS]-[L]-[PS] strands 315. Single stranded ligation may be mediated by an enzyme, such as a CIRCLIGA SE enzyme (discussed in more detail below).

In Step D′, the adapter portion of [T]-[PS]-[L]-[PS] strands 315 may be dephosphorylated, e.g., using an enzyme such as T4 PNK (New England Biolabs, Inc., Ipswich, Massachusetts) producing dephosphorylated strand 320. T4 PNK prepares the fragment ends for circularization by ligation in Step F by leaving a 3′-OH end of a bisulfite-converted ssDNA fragment. Step D′ may also include heat deactivation of the dephosphorylating enzyme prior to moving to circularization in Step F. As described above for circularization of [PS]-[L]-[PS]-[T′] 235 strands in Step F, [T]-[PS]-[L]-[PS] strands 315 are similarly circularized in Step F, yielding circularized [T]-[PS]-[L]-[PS] templates 240. Templates 240 represent both circularized [PS]-[L]-[PS]-[T′]235 strands and [T]-[PS]-[L]-[PS] strands 315.

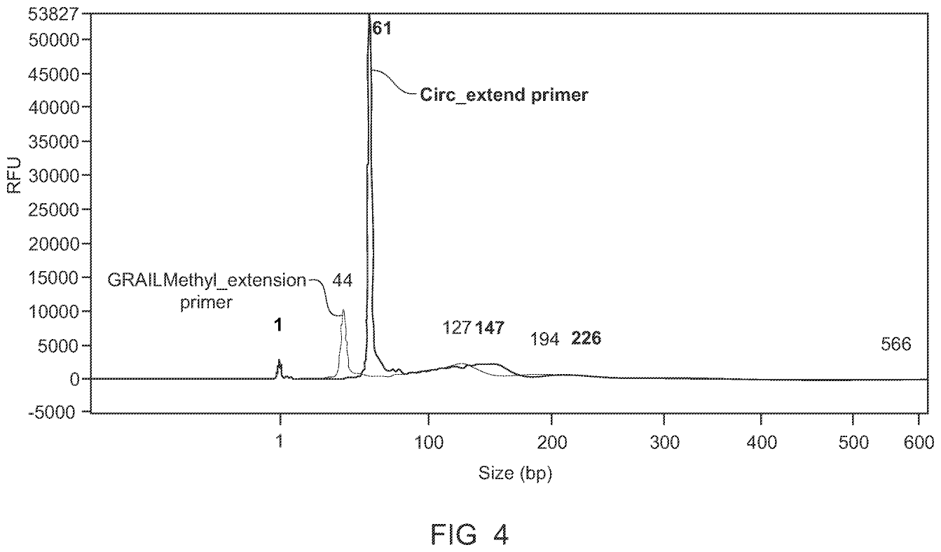

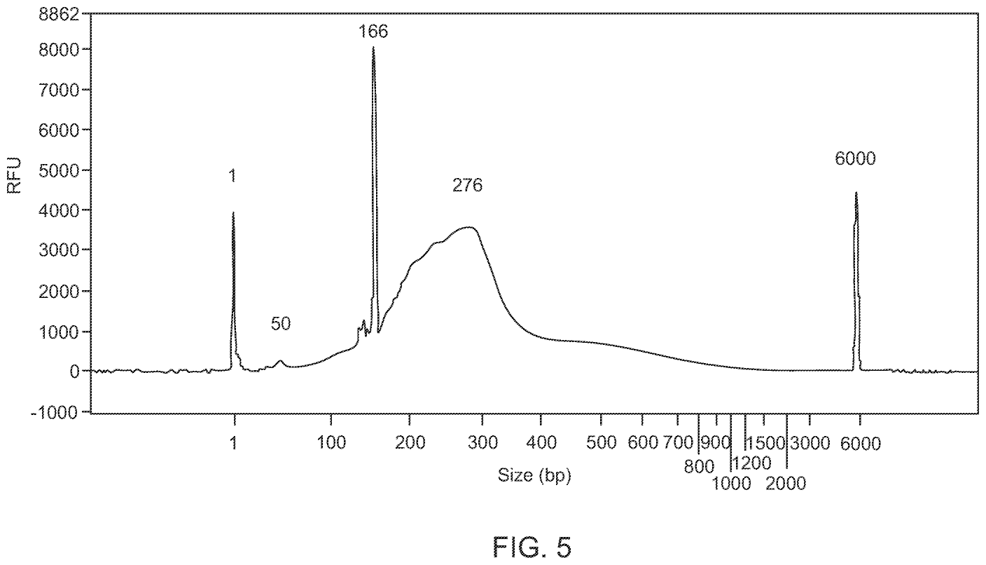

Experimental results of various embodiments of the disclosed subject matter are described in Example 1 and illustrated in FIG. 4 and FIG. 5. FIG. 4 is a graph of a High Sensitivity NGS Fragment Analyzer run illustrating a comparison of cfDNA prepared in an amplification method according to FIGS. 2A and 2B using either the [PS1]-[L]-[PS2] strand 102 as an extension primer (designated “Circ extend primer”) or a traditional extension primer where the cfDNA is not circularized (designated “GrailMethylSeq”), and where the cfDNA has received only one of two clean up steps. FIG. 5 is a graph of a High Sensitivity NGS Fragment Analyzer run of a methylation library that was prepared by a circularization approach according to FIGS. 2A and 2B using the Circ extend primer [PS1]-[L]-[PS2] strand 102.

### Input Sample

In the methods of the disclosed subject matter, the input sample includes fragmented DNA. The fragmented DNA may be fragmented using various laboratory techniques. The fragmented DNA may be naturally fragmented, e.g., cfDNA. The fragmented DNA may represent any subset of a genome, including a whole genome, or even multiple genomes or subsets of multiple genomes.

The source of the sample may be any source of DNA. For example, the sample source may be a biological organism or an environmental sample. Where the source is a biological organism, the source may be tissues, cells, fluids or other substances. The sample may be fresh or may be preserved. Preferably the subject is a human or other animal. Samples or input samples may in one aspect of the disclosed subject matter be pooled from multiple sources and/or multiple subjects.

In one aspect of the disclosed subject matter, the sample is from a subject known to have or suspected of having a certain disease. In one aspect of the disclosed subject matter, the sample is from a subject not known to have or suspected of having a certain disease (e.g., a control subject in a study or a subject undergoing screening for a disease).

In one version of the disclosed subject matter, the sample is from a subject known to have or suspected of having a cancer. In one aspect of the disclosed subject matter, the sample is from a subject not known to have or suspected of having cancer (e.g., a control subject in a study or a subject undergoing screening for cancer).

In some embodiments of the disclosed subject matter, the sample is from a tumor or a suspected tumor. In one aspect of the disclosed subject matter, the sample is a tissue sample that may be a cancer tissue or is suspected of being a cancer tissue. In one aspect of the disclosed subject matter, the sample is a tissue sample that may be a stage I, II, III, or IV cancer.

In one aspect of the disclosed subject matter, the sample is a bodily fluid or extracellular bodily substance. In some versions of the disclosed subject matter, the bodily fluid or extracellular bodily substance is selected from the group consisting of whole blood, a blood fraction, serum, and plasma. In one aspect of the disclosed subject matter, the bodily fluid or extracellular bodily substance is selected from aqueous humor, ascites, bile, cerebral spinal fluid, chyle, gastric juices, intestinal juices, lymphatic fluid, pancreatic juices, pericardial fluid, peritoneal fluid, pleural fluid, saliva, spinal fluid, sputum, stool or other intestinal waste fluids, sweat, tears, and/or urine.

According to one version of the disclosed subject matter, the input sample is cfNA or cfDNA obtained from a bodily fluid or other bodily substance. In one aspect of the disclosed subject matter the cfNA or cfDNA originate from healthy cells. In one aspect of the disclosed subject matter the cfNA or cfDNA originate from diseased cells, such as cancer cells.

Circularization and ssDNA Ligation

As noted above, in certain steps of the methods of the disclosed subject matter a nucleic acid strand is circularized (see Step F, illustrated in FIG. 2B). In certain steps of the methods of the disclosed subject matter a single stranded nucleic acid strand is circularized. In other steps ssDNA molecules are ligated.

In one embodiment, T4 DNA ligase may be used to circularize single-stranded DNA. The ligation reaction can be, for example, a “standard” T4 DNA ligase reaction or a modified version of the standard reaction designed to prevent intermolecular polymerization and promote intramolecular cyclization as described by An, R., et. al., Nucleic Acids Research (2017) 45(15): e139, which is incorporated herein by reference in its entirety. The ligation reactions may be performed using, for example, T4 DNA ligase and T4 DNA ligase buffer available from Thermo Scientific (Pittsburgh, PA).

In one example, the standard T4 DNA ligase reaction (20 uL) may be performed using 1 μM linear single-stranded DNA, IX T4 DNA ligase buffer (10 mM Mg2+, 500 μM ATP, 10 mM dithiothreitol (DTT), and 40 mM Tris-HCl), “splint” DNA (2 μM; length 12 nucleotides), and 5 U T4 DNA ligase. The ligation reaction may be performed at 20° C. for 12 hours, and the reaction terminated by heating the reaction mixture at 65° C. for 10 minutes.

In one example, a modification of the standard reaction may be performed using T4 DNA ligase buffer diluted to 0.05× (0.5 mM Mg2+, 25 μM ATP, 0.5 mM DTT, and 2 mM Tris-HCl).

In another example, a modification of the standard reaction may be performed using T4 DNA ligase buffer diluted to 0.05× (0.5 mM Mg2+, 25 μM ATP, 0.5 mM DTT, and 2 mM Tris-HCl) and step-wise addition of the linear single-stranded DNA to the ligation reaction at predetermined time intervals.

In one aspect, DNA may be circularized by modifying the 5′ end of the DNA strand with a phosphate group to make it circularize-able to the 3′ end of the same strand. The scaffold strand may be folded to join its two ends together by a splint strand that joins both ends together by annealing, e.g., at 95° C. to 25° C. for 4-h followed by ligating the two ends using T4 DNA ligase (10 μL, 350 U/μL) in a TE buffer having 66 mM Tris-HCl (pH 7.6), 6.6 mM MgC12, 10 mM dithiothreitol (DTT), 0.1 mM ATP and 3.3 μM Na432P2O7 followed by the incubation at 16° C. for 16 h in an alcohol bath machine. T4-DNA/Ligase may be heat-denatured at 65° C. for 10 min followed by sudden cooling in an ice-bath for 5 min.

In one embodiment, T4 RNA ligase may be used to circularize single-stranded DNA. In one example, single-stranded DNA can be circularized in a ligation reaction using T4 RNA ligase, reaction buffer, and bovine serum albumin (BSA) available from Thermo Scientific (Thermo Fisher Scientific, Waltham, MA) following the manufacturer's recommended protocol, which is incorporated herein by reference in its entirety.

In another example, TS2126 RNA ligase 1 may be used to catalyze intramolecular single stranded DNA ligation (circularization) in an ATP-dependent manner as described by Blondal et al., Nucleic Acids Res. (2005) 33(1): 135-142, which is incorporated herein by reference in its entirety.

CIRCLIGASE ssDNA Ligase is a thermostable ATP-dependent ligase that catalyzes intramolecular ligation (i.e., circularization) of single-stranded DNA (ssDNA) substrates that have both a 5′-monophosphate and a 3′-hydroxyl group. CIRCLIGASE II ssDNA Ligase is an ATP-independent, non-catalytic thermostable ligase that catalyzes the intramolecular ligation (i.e., circularization) of ssDNA templates. Both enzymes are available from Lucigen Corp. (Middleton, WI). The CIRCLIGASE ssDNA Ligase kit or CIRCLIGASE II ssDNA Ligase kit (EPICENTRE Biotechnologies, Madison, WI; available from Lucigen Corp., Middleton, WI) may be used to circularize single-stranded DNA following the manufacturer's recommended protocol, which is incorporated herein by reference in its entirety. The Lucigen Corp, manuals for CIRCLIGASE ssDNA Ligase (M A222E-CIRCLIGASE ssDNA Ligase, March 2019) and CIRCLIGASE II ssDNA Ligase (MA298E-CIRCLIGASE II ssDNA Ligase. July 2019) are incorporated herein in their entireties.

In one embodiment, CIRCLIGASE may be used to circularize single-stranded DNA. In one example, the CIRCLIGASE ssDNA Ligase kit (EPICENTRE Biotechnologies, Madison, WI; available from Lucigen Corp., Middleton, WI) may be used to circularize single-stranded DNA following the manufacturer's recommended protocol, which is incorporated herein by reference in its entirety. In one example, the ligation reaction may be performed using 10 pmol single-stranded DNA, 2 μL of CircLigase 10× reaction buffer, 1 μL of 1 mM ATP, 1 μL of 50 mM MnC12, 1 μL of CircLigase ssDNA Ligase (100 U) to yield a 20 μL total reaction volume. The ligation reaction may be incubated at 60° C. for 1 hour and the reaction terminated by heating the reaction mixture at 80° C. for 10 minutes to inactivate the ligase.

In another example, the CIRCLIGASE II ssDNA Ligase kit (EPICENTRE Biotechnologies, Madison, WI; available from Lucigen Corp., Middleton, WI) may be used to circularize single-stranded DNA following the manufacturer's recommended protocol, which is incorporated herein by reference in its entirety. In one example, the ligation reaction may be performed using 10 pmol single-stranded DNA, 2 μL, of CIRCLIGASE II 10× reaction buffer, 1 μL of 50 mM MnC12, 4 μL of 5 M betaine (optional), 1 μL of CIRCLIGASE II ssDNA Ligase (100 U) to yield a 20 pL total reaction volume. The ligation reaction may be incubated at 60° C. for 1 hour and the reaction terminated by heating the reaction mixture at 80° C. for 10 minutes to inactivate the ligase.

In another example, DNA may be circularized by resuspending DNA in 4.5 μL of circularization mix (final volume in 5 μL: IX CIRCLIGASE buffer, 50 μM ATP, and 2.5 mM MnCL) and adding 0.5 μL CIRCLIGASE. Circularization may be performed for 1 hour at 60° C. for 1 hour and the reaction terminated by heating the reaction mixture at 80° C. for 10 minutes to inactivate the ligase.

CIRCLIGASE ssDNA Ligase and CIRCLIGASE II ssDNA Ligase are also useful for ligating linear ssDNA molecules, e.g., as shown with respect to Step C of FIG. 2A and Step C′ of FIG. 3. Any known protocol can be used. An illustrative example is provided in Gansauge, et al., Single-stranded DNA library preparation from highly degraded DNA using T4 DNA ligase, Nucleic Acids Res. 2017 Jun. 2; 45 (10): e79, the disclosure of which is incorporated herein by reference for its teaching concerning methods of ssDNA ligation.

### Conversion of Uracils

As noted above, in certain steps of the methods of the disclosed subject matter, unmethylated cytosines may be converted to uracils.

A variety of kits are commercially available for this purpose. Examples include EPIMARK Bisulfite Conversion Kit (New England Biolabs Ltd., Ipswich, Massachusetts); ACTIVEMOTIF Bisulfite Conversion Kit (Active Motif, Inc., Carlsbad, California); EPITECT Bisulfite Kits (QIAGEN Ltd., Hilden, Germany); EZ DNA Methylation-Lightning Kit (Zymo Research Corp., Irvine, California); NEBNEXT Enzymatic Methyl-seq (EM-SEQ) (New England Biolabs, Inc., Ipswich, Massachusetts). The product literature of these kits is incorporated herein by reference.

In one aspect, the DNA fragments are denatured and treated with a bisulfite. The denaturation and bisulfite treatment steps may be in a single reaction or may be conducted in sequential reactions. Bisulfite treatment modifies unmethylated cytosines with a sulfite. After conversion, the DNA may be deaminated to convert to uracil. For example, the DNA may be desalted and incubated at alkaline pH resulting in deamination and conversion to uracil.

In one aspect of the disclosed subject matter, the DNA fragments may be denatured using NaOH at a final concentration of about 0.3 M and treated with sodium bisulfite or sodium metabisulfite at a final concentration of about 2M (pH between about 5 and 6) at 55° C. for 4-16 hours. After conversion, the DNA may be desalted followed by desulfonation by incubating the DNA at alkaline pH at room temperature.

In another aspect of the disclosed subject matter, the conversion of unmethylated cytosines to uracils makes use of enzymatic techniques. For example, certain cytosine deaminases are known for deaminating cytosine bases to uracil in single-stranded DNA.

In another aspect of the disclosed subject matter, the cytosine deamninase is APOBEC. APOBEC also deaminates 5 mC and 5 hmC, so in order to detect 5 mC and 5 hmC, these methods use techniques to block deamination of 5 mC and/or 5 hmC. For example, using EM-SEQ (New England Biolabs, Ipswich, Massachusetts) TET2 and an oxidation enhancer can be used to modify 5 mC and 5 hmC to forms that are not substrates for APOBEC. The TET2 enzyme converts 5 mC to 5 caC, and the oxidation enhancer converts 5 hmC to 5 ghmC. The NEBNEXT Enzymatic Methyl-seq (EM-SEQ) product literature is incorporated herein by reference.

In another aspect of the disclosed subject matter, 5 hmC is selectively converted so that it can be identified separately from 5 mC. For example, APOBEC-coupled epigenetic sequencing (ACE-seq) relies on enzymatic conversion to detect 5 hmC. With this method, T4-BGT glucosylates 5 hmC to 5 ghmC and protects it from deamination by APOBEC3A. Cytosine and 5 mC are deaminated by APOBEC3A and sequenced as thymine.

In another aspect of the disclosed subject matter, oxidative bisulfite sequencing (oxBS) is used to distinguish between 5 mC and 5 hmC. The oxidation reagent potassium perruthenate converts 5 hmC to 5-formylcytosine (5 fC) and subsequent sodium bisulfite treatment deaminates 5 fC to uracil. 5 mC remains unchanged and can therefore be identified using this method.

In another aspect of the disclosed subject matter, fragmented DNA is treated with T4-BGT which protects 5 hmC by glucosylation. The enzyme mTET1 is then used to oxidize 5 mC to 5 hmC, and T4-BGT labels the newly formed 5 hmC using a modified glucose moiety (6-N3-glucose).

### Strand Denaturation

In one aspect of the disclosed subject matter, the strands are denatured prior to conducting the conversion reaction. Denaturation may, for example, be accomplished by incubation at elevated temperatures, e.g., about 98° C., and/or exposure to a base, such as sodium hydroxide.

### Cleanup Steps

The method of the disclosed subject matter may benefit from cleanup steps at various stages to prepare the reactants for subsequent steps. In one example, a bead-based cleanup protocol is performed, e.g., a SPRI-cleanup protocol.

The following examples are offered by way of illustration and not by way of limitation.

### EXAMPLES

### Example 1

A single stranded nucleic acid molecule containing an organic linker (also referred to as “Circ extend primer”) was designed and tested for use in a circularization and extension approach to preparing methylation libraries. The following experimental procedures describe an approach for making next generation sequencing (NGS) targeted methylation libraries from cfDNA where the DNA is circularized prior to amplification. This circularization methyl-seq library preparation workflow design is illustrated in FIGS. 2A and 2B.

### Circ_Extend Primer Design

The following Circ Extend primer (strand 102 in FIG. 1B) was used: /5Phos/CGACAGGTTCAGAGTTCCT AC AGGTCCGACGATC/iSp 18/C ACTC A/iSp 18/GTGACTG GAGTTCAGACGTGTGCTCTTCCGATCT-3′OH. In this extension primer, the iSp!8/CACTCA/iSp18 portion of the molecule is the linker 120 illustrated in FIG. 1B. The term “iSP18” designates Int Spacer 18, a hexa-ethyleneglycol molecule available from Integrated DNA Technologies, Inc. (Coraville, IA).

### Input Sample

Cell-free DNA (cfDNA) present within the plasma fraction of whole blood.

### Conversion of Uracils

The EZ DNA METHYLATION-LIGHTNING Kit Manual Version 1.0.4 (Zymo Research Corp., Irvine, California) was used to convert unmethylated cytosines in the cfDNA to uracils. Fragments were analyzed by capillary electrophoresis using the Advanced Analytical Technologies High Sensitivity NGS Fragment Analysis Kit (1 bp-6000 bp) User Guide for DNF-474-0500 and DNF-474-1000.

### Purification of NGS Libraries

NGS libraries were purified during library construction and before sequencing using Solid Phase Reversible Immobilisation (SPRI). The method relies on carboxyl-coated magnetic particle s/beads that reversibly bind DNA in the presence of polyethylene glycol (PEG) and salt.

### Reagents and Materials

| | Vendor/ |
|---|---|
| Description | Part Number |
| cfDNA (Input B) | Grail/G0315 |
| EZ DNA Methylation-Lightning ™ Kit | Zymo Research/D5031 |
| UltraPure ™ IM Tris-HCI, pH 8.0 | Thermo Fisher/ |
| | 15568025 |
| Ethy 1 alcohol, Pure | Sigma/E7023-6X500ML |
| SPRI bead | Grail/G0673 |
| UltraPure ™ DNase/ | Thermo Fisher/ |
| RNase-Free Distilled Water | 10977023 |
| Low EDT A TE buffer | Thermo Fisher/ |
| | 12090015 |
| FastAP Thermosensitive | Thermo Fisher/EF0654 |
| Alkaline Phosphatase | |
| KAP A HiFi HotStart Ready Mix (2X) | Roche/ |
| | 07958935001 |
| TS2126 ssLigase (100 U/uL) | Grail/G0637 |
| ssLigase Reaction Buffer (10X) | Grail/G0670 |
| MnC12 | Grail/G0669 |
| VeraSeq Ultra Ready Mix (2X) | Grail/G0709 |
| PEG4000 (50%) (From T4 DNA ligase kit) | Thermo Fisher/EL0011 |
| Fragment Analyzer Kit | Agilent/DNF-473 |

### Polyethylene Glycol (PEG) Preparation

A 42% (final concentration) preparation oF PEG was prepared by combining 1000 μl PEG (50%) with 190.5 μL ULTRAPURE DNase/RNase-Free Water.

### Library Preparation Oligonucleotide Reagents

The oligonucleotides used in the experiments were dissolved in low ED TA TE buffer and are shown in the Table below.

| | | | | Working |
|---|---|---|---|---|
| | | | Stock | Dilution |
| Name | Description | Sequence (5′-3′) | (μM) | (μM) |
| GMS Adapter 1 | Blocked 20 base | /5Phos/AGATCGGAAGAGCACACGTC/3AmMO/ | 1000 | 10 |
| | single-stranded | | | |
| | adapter | | | |
| | (Dual HPLC | | | |
| | purified) | | | |
| Circ_extend | Extension primer | /5Phos/CGACAGGTTCAGAGTTCCTACAGGT | 100 | 100 |
| primer | (HPLC purified) | CCGACGATC/iSp 18/CACTCNiSp18/GTGACT | | |
| | | GGAGTTCAGACGTGTGCTCTTCCGATCT | | |
| P5_G00li7_ | PCR primer | AATGATACGGCGACCACCGAGATCTACAT | 100 | 50 |
| circPCR | | CGACATGGAGATCGTCGGACCTGTAGGAA | | |
| | | CTCTGAACCTGTCG | | |
| P5_G00li7_ | PCR primer | GATCGGAAGAGCACACGTCTGAACTCCAG | 100 | 50 |
| circPCR | | TCACAGTGGTAACTATCTCGTATGCCGTCT | | |
| | | TCTGCTTG | | |
| Circ_seq | Sequencing | GATCGTCGGACCTGTAGGAACTCTGAACCT | 100 | 100 |
| | primer | GTCG | | |

* = Phosphorothioate linkage
ddT = dideoxy Thymine
3AmM0 = 3′ amino modifier, available from Integrated DNA Technologies, Inc.
(Coraville, IA
iSp18 = Int Spacer 18, a hexa-ethyleneglycol molecule available from Integrated DNA Technologies, Inc. (Coraville, IA)

### General Procedures

All reaction mixes were mixed by pulse vortexing before adding the enzymes. Mixing after the enzyme addition was only performed by gentle flicking of the tube followed by a quick spin, except in the section of Ligation of First Adapter (where the reaction contains 20% PEG-4000 and vortexing is necessary for proper mixing). Low-Bind pipette tips were used throughout the protocol.

### Bisulfite Conversion Preparation

96 mL of 100% ethanol was added to the 24 mL M-Wash Buffer concentrate (D5031) before use. ZYMO-SPIN IC Columns were checked to make sure there was no visible damage on the column or matrix before use. Lightning Conversion Reagent was mixed by vortexing and quickly spun down.

### Bisulfite Conversion

A total of 25 ng Input ctDNA was placed in a 0.2 mL PCR strip tube. Sample volume was adjusted to 20 μL with nuclease free water as needed. 130 μL of Lightning Conversion Reagent was added to DNA sample. Mixed by vortexing, then centrifuged briefly to ensure there were no droplets in the cap or sides of the tube. EZ lightning program was started on the thermocycler. When temperature reaches 90° C., the tubes were carefully placed in the thermocycler. Cycle program was as follows: 98° C. for 8 minutes; 54° C. for 60 minutes; and 4° C. hold.

### Column Clean Up

ZYMO-SPIN IC Column was set up into a provided collection tube. 600 μL of M-Binding Buffer was added to the column. The DNA tubes were removed from the thermocycler carefully. The sample was loaded into the ZYMO-SPIN IC Column containing the M-Binding Buffer. Mixed by pipetting up and down. Centrifuged at full speed (>10,000×g) for 30 seconds. The flow-through was discarded. 100 μL of M-Wash Buffer was added to the column and it was centrifuged at full speed for 30 seconds. The flow-through was discarded. 200 μL of L-Desulphonation Buffer was added to the column and let stand at room temperature for 20 minutes. Then it was centrifuged at full speed for 30 seconds. The flow-through was discarded and changed to new collection tubes. 200 μL of M-Wash Buffer was added to the column. Column was centrifuged at full speed for 30 seconds. Another 200 μL of M-Wash Buffer was added to the column and centrifuged at full speed for 30 seconds. The column was placed in a new collection tube and centrifuged at full speed for 30 seconds. The column was placed into 1.5 mL Eppendorf Lo-Bind tube, 2 μL of M-Elution Buffer added directly to the column matrix, then incubated at room temperature for 5 minutes. Centrifuged for 30 seconds at full speed to elute the DNA.

### Dephosphorylation & Heat Denaturation

11 μL bisulfite treated DNA was transferred to 0.2 mL strip tubes and the following reaction reagent mix set up in tubes on ice:

| | Volume (μL) per |
|---|---|
| Reagents | Sample |
| ssLigase Reaction Buffer | 3 |
| (10x) | |
| MnCl₂ (50 mM) | 2 |
| FastAP (1 U/μL) | 1 |

6 μL master-mix was added to the bisulfite treated DNA and mixed by flicking gently then a quick spin. The tubes were incubated in a thermocycler with a pre-heated lid (105° C.) at: 37° C. for 10 minutes; 95° C. for 2 minutes; and then moved immediately to an ice-water bath and let sit for 1 minute. After a quick spin, Proceed to Ligation of First Adapter.

### Ligation of First Adapter

The purified GMS Adapter 1 was thawed on ice. The following reaction reagent was set up, mixing by vortex:

| | Volume (μL) per |
|---|---|
| Reagents | Sample |
| PEG-4000(42%) | 20 |
| GMS Adapterl (10 μM) | 1 |
| TS2126 ssLigase | 2 |
| (100 U/μL) | |

23 μL master-mix was added to the 17 μL dephosphorylated DNA. Mix by brief vortexing and a quick spin. The tubes were incubated in a thermocycler with a pre-heated lid (105° C.) at: 58° C. for 1 hour.

### Ssdna SprI Purification: (SprI 1)

The following procedure was performed:

1. Diluted SPRI preparation:

| | Volume (μL) per |
|---|---|
| Reagents | Sample |
| low EDTA TE | 11 |
| buffer | |
| SPRI beads | 72 |

2. Add 83 μL of the diluted SPRI beads to the 40 μL ligated DNA. Mix by pipetting up and down.

3. Incubate at room temperature for 20 minutes.

4. After a quick spin, place the tubes on a magnetic stand for 5 minutes. Discard the supernatant without touching the beads.

5. Wash 2 times with 200 μL 80% fresh made Ethanol.

6. After a quick spin, use a 20 μL pipette to remove residual ethanol. Air-dry the pellet on the magnetic stand for 3 minutes.

7. Remove the tubes from the magnetic stand. Add 24 μL RSB, mix then incubate at RT for 2 minutes. After a quick spin, place the tube on a magnetic stand for 5 minutes.

8. Transfer 24 μL to the “Linear amplification” reaction.

### Linear Amplification

The Linear amplification reaction was set up and performed as follows:

| | Volume (μL) per |
|---|---|
| Reagents | Sample |
| Ligated DNA | 24 |
| VeraSeq Ultra Ready Mix | 25 |
| (2X) | |
| Circ_extend (100 μM) | 1 |

Incubate the tubes in a thermocycler with a pre-heated lid (105° C.) at:
1 cycle: 98° C. for 30 seconds
20 cycles:
98° C. for 10 seconds
62° C. for 30 seconds
72° C. for 30 seconds
1 cycle: 72° C. for 10 minutes
2 4° C. hold

### Ssdna Double SPRI Purification: (SPRI 2)

The following protocol was followed:

1. Prepare SPRI PEG mix for IX SPRI purification:

| | Volume (μL) per |
|---|---|
| Reagents | Sample |
| PEG (50%) | 27 |
| SPRI | 50 |

2. Add 77 μL to 50 μL amplified DNA. Mix by pipetting up and down. Incubate at RT for 20 minutes.

3. After a quick spin, place the tubes on a magnetic stand for 5 minutes. Discard supernatants.

4. Wash 2 times with 200 μL 80% ethanol.

5. After a quick spin, use a 20 μL pipette to remove residual ethanol Air-dry the pellet on the magnetic stand for 3 minutes.

6. Remove the tubes from the magnetic stand. Add 50 μL RSB mix. Incubate at RT for 2 minutes.

7. After a quick spin, place the tubes on a magnetic stand for 5 minutes.

8. Transfer 50 μL to a new tube for 0.9× SPRI purification.

9. Prepare SPRI PEG mix for 0.9× SPRI purification

| | Volume (μL) per |
|---|---|
| Reagents | Sample |
| PEG (50%) | 25 |
| SPRI | 45 |

10. Add 70 μL to 50 μL amplified DNA. Mix by pipetting up and down. Incubate at RT for 20 minutes.

11. After a quick spin, place the tubes on a magnetic stand for 5 minutes. Discard supernatants.

12. Wash 2 times with 200 μL 80% ethanol.

13. After a quick spin, use a 20 μL pipette to remove residual ethanol. Air-dry the pellet on the magnetic stand for 3 minutes.

14. Remove the tubes from the magnetic stand. Add 26 μL RSB mix. Incubate at RT for 2 minutes.

15. After a quick spin, place the tubes on a magnetic stand for 5 minutes.

16. Transfer 25 μL to the “Denature and Circularization” reaction.

### Denature and Circularization

The following procedure was followed:

1. Denature the ssDNA by heating in a thermocycler with a pre-heated lid (105° C.) at 95° C. for 2 minutes

2. Move immediately to ice-water bath and let sit for 1 minute

3. Set up the following reaction on ice

| | Volume (μL) per |
|---|---|
| Reagents | Sample |
| Denatured ssDNA | 25 |
| ssLigase Reaction Buffer (10x) | 3 |
| MnCh (50 mM) | 1.5 |
| TS2126 ssLigase (100 U/μL) | 1.5 |

4. Mix gently followed by a quick spin. The final volume is 31 μL.

5. Incubate the tubes in a thermocycler with a pre-heated lid (105° C.) at:
60° C. for 60 minutes.
80° C. for 10 minutes.
4° C.

### Library Amplification and Indexing

The following procedure was followed: Set up the following reaction on ice:

| | Volume (μL) per |
|---|---|
| Reagents | Sample |
| Circularization ligated DNA | 31 |
| KAP A HiFi HotStart Ready Mix | 40 |
| (2X) | |
| P7_G001i7_circPCR (50 μM) | 5 |
| P5_G001i5_circPCR (50 μM) | 5 |

1. Mix gently followed by a quick spin. The final volume is 81 μL.

2. Incubate in a thermocycler with a pre-heated lid (105° C.) at:
1 cycle: 98° C. for 45 seconds.
14 cycles:
98° C. for 15 seconds.
60° C. for 30 seconds.
72° C. for 30 seconds.
1 cycle: 72° C. for 1 minute.
4° C.

### Amplified Library SPRI Beads Clean Up (SPRI 4)

The following procedure was followed:

1. Add 114 μL SPRI beads. (1.4×). Mix by pipetting up and down.

2. Incubate at RT for 10 minutes. After a quick spin, place the tubes on a magnetic stand for 5 minutes. Remove and discard supernatant.

3. Wash 2 times with 200 ul 80% ethanol.

4. After a quick spin, use a 20 μL pipette to remove residual ethanol. Air-dry the pellet on the magnetic stand for 3 minutes.

5. Add 23 μL RSB in the tube. Mix and incubate at RT for 2 minutes. Quick spin, place the tubes on a magnetic stand for 5 minutes,

6. Transfer 20 μL to a new tube. This is the methylation library.

### Library QC

The following procedure was followed:

1. Prepare 1:100 library dilution, then add 2 μL of the library on the Fragment Analyzer using the High Sensitivity NGS kit. 2. Select the 175-5,000 bp range to quantify the amount of library.

### Results

FIG. 4 is a graph illustrating the results of using the Circ extend primer in the amplification experiment described above. Circ extend primer was used in place of a traditional extension primer (designated “GrailMethylSeq”) in which the DNA is not circularized in the amplification method. After the first 1× SPRI clean up, the DNA was run on a High Sensitivity NGS Fragment Analyzer for comparison with DNA prepared using the GrailMethylSeq extension primer.

FIG. 5 is a graph illustrating methylation library DNA prepared by the circularization approach described herein using Circ extend primer. The library DNA was diluted 1:100 and run on a High Sensitivity NGS Fragment Analyzer and the results are shown in FIG. 5.

1. A circularized nucleic acid template having the structure [T]-[PS1]-[L]-[PS2] or [PS1-L]-PS2[T′], wherein: (a) T and T′ each is a target nucleic acid sequence; (b) each of PS1 and PS2 is a nucleic acid primer site; (c) L is a linker comprising a primer extension reaction terminating organic molecule; and (d) the structure is circularized by binding a 5′ end thereof to a 3′ end thereof.

2. (canceled)

3. A method of amplifying a target sequence, the method comprising: (a) providing circularized template of claim 1; (b) binding to PS1 a primer complimentary to PS1 and binding to PS2 a primer complimentary to PS2; and (c) copying the target sequence by a primer extension reaction using a polymerase.

4. (canceled)

5. A method of making a circularized template of claim 1, the method comprising: (a) providing a [PS1]-[L]-[PS2] strand, wherein each of PS1 and PS2 is a nucleic acid primer site and L is a linker comprising a primer extension reaction terminating organic molecule; (b) providing a [T] nucleic acid; (c) ligating the [PS1]-[L]-[PS2] strand to the [T] strand to produce a [T]-[PS1]-[L]-[PS2] strand; (d) circularizing [T]-[PS1]-[L]-[PS2] strand to produce a circularized [T]-[PS1]-[L]-[PS2],

6. A method of making a circularized template of claim 1, the method comprising: (a) providing a [T] strand; (b) providing a [PS1]-[L]-[PS2] strand, wherein each of PS1 and PS2 is a nucleic acid primer site and L is a linker comprising a primer extension reaction terminating organic molecule; (c) ligating to a 3′ end of the [T] strand a primer site [PS2′] strand complementary to the [PS2] of the [PS1-[L]-[PS2] strand; (d) annealing the [PS1]-[L]-[PS2] strand to the ligated [T]-[PS2′]; (d) extending the [PS1]-[L]-[PS2]strand by a primer extension reaction using a polymerase to produce a [PS1]-[L]-[PS2]-[T′] strand], wherein [T] is a complement of the [T] strand; and (e) circularizing the [PS1]-[L]-[PS2]-[T′] strand to produce a circularized [PS1]-[L]-[PS2]-[T′].

7-8. (canceled)

9. The circularized template of claim 1, wherein L comprises a polyalkylene glycol or a polyethylene glycol.

10. (canceled)

11. The circularized template of claim 9, wherein L comprises a polyalkylene glycol or a polyethylene glycol terminated with a phosphate group.

12. (canceled)

13. The circularized template of claim 1, wherein L comprises one or multiple insertions of a polyalkylene glycol, a polyalkylene glycol terminated with a phosphate group, a polyethylene glycol, a polyethylene glycol terminated with a phosphate group, a phosphonamidite, a glycol, a 1′,2′-Dideoxyribose, or a triethylene glycol terminated with a phosphate group, or any combination thereof.

14. The circularized template of claim 13, wherein the multiple insertions are connected through one or more intervening oligonucleotides.

15. The circularized template of claim 14, wherein the one or more intervening oligonucleotides range in length from 2 nucleotides in length to 100 nucleotides in length.

16. The circularized template of claim 14, wherein L comprises two insertions of the polyethylene glycol terminated with a phosphate group connected through the intervening oligonucleotide.

17. The circularized template of claim 16, wherein the length of the intervening oligonucleotide is six or more nucleotides.

18. The circularized template of claim 1, wherein the target comprises a single-stranded DNA molecule in which cytosines have been modified or converted.

19. The circularized template of claim 18, wherein the cytosines have been converted to uracils.

20. The method of claim 3, wherein the primer complementary to PS1 comprises a structure 5′ to 3′ [ADAPT]-[IND]-[SEQ-PRIMER]-[TARG-PRIMER], wherein [ADAPT] is an adapter, [IND] is a unique sample identifier sequence, [SEQ-PRIM ER] is a sequencing primer, [TARG-PRIMER] is a target primer complementary to a target nucleic acid sequence in the circularized [T]-[PS]-[L]-[PS] or [PS]-[L]-[PS]-[T′] template; and wherein the primer complementary to PS2 comprises a structure 5′ to 3′ [ADAPT]-[IND]-[SEQ-PRIMER]-[TARG-PRIMER], wherein [ADAPT] is an adapter, [IND] is a unique sample identifier sequence, [SEQ-PRIMER] is a sequencing primer, and [TARG-PRIMER] is a target primer complementary to a target sequence in the circularized [T]-[PS]-[L]-[PS] or [PS]-[L]-[PS]-[T′] template.

21. The method of claim 3, wherein the target comprises fragmented DNA.

22. The method of claim 21, wherein the fragmented DNA is cell free nucleic acid (cfNA).

23. The method of claim 22, wherein the cfNA is cell free DNA (cfDNA).

24. The method of claim 22, wherein the cfNA is obtained from a bodily fluid or other bodily substance.

25. (canceled)

26. The method of claim 22, wherein the cfNA originates from diseased cells.

27-41. (canceled)


SEQUENCE LISTING (4 sequences)

SEQ ID NO: 1 - DNA, 20 bp, Artificial Sequence
  Adapter
aatgatacggcgaccaccga

SEQ ID NO: 2 - DNA, 33 bp, Artificial Sequence
  Sequencing primer
acactctttccctacacgacgctcttccgatct

SEQ ID NO: 3 - DNA, 21 bp, Artificial Sequence
  Adapter
caagcagaagacggcatacga

SEQ ID NO: 4 - DNA, 34 bp, Artificial Sequence
  Sequencing primer
gtgactggagttcagacgtgtgctcttccgatct